(19) United States
(12) Patent Application Publication
(10) Pub. No.: US 2008/0010697 A1
(43) Pub. Date: Jan. 10, 2008

(54) Methods of Expressing Heterologous Protein in Plant Seeds Using Monocot Non Seed-Storage Protein Promoters

(76) Inventors: Daichang Yang, Wuhan (CN); Kevin Hennegan, Denver, CO (US); Ning Huang, Davis, CA (US)

Correspondence Address: ARENT FOX LLP, 1050 CONNECTICUT AVENUE, N.W., SUITE 400, WASHINGTON, DC, 20036, US

(21) Appl. No.: 10/584,225

(22) PCT Filed: Dec. 23, 2003
(86) PCT No.: PCT/US03/39107

§ 371 Date: Jul. 13, 2007

(51) Int. Cl. A01H 1/02; C12N 15/87

(52) U.S. Cl. 800/260; 800/278; 800/288

(57) ABSTRACT

The invention is directed to expression of non-plant proteins in rice plants. Expression is optimized by use of a non-rice promoter of a monocot protein gene and its corresponding signal peptide for expression of the non-plant protein in rice plant at high yields. The invention is useful for making human proteins, polypeptides and peptides in rice seeds. The expressed protein product can be isolated from the rice seed for administration to humans or other animals.

### FIELD OF THE INVENTION

The present invention relates to methods of expressing heterologous proteins in the seeds of angiosperm plants such as monocots, e.g. rice plants. Expression of the heterologous proteins can be optimized by using monocot promoters and signal sequences for expression of proteins in angiosperm, preferably monocot seeds.

### BACKGROUND OF THE INVENTION

Many human proteins are in short supply due to the large quantities required of the proteins for therapeutic uses or due to the large demand of these proteins by the world population. Expression of the human proteins in plants is a potential way of meeting the increased demand of the proteins. Plant expression of the human proteins can be more desirable than expression of the human proteins in a prokaryotic microorganism due to potential differences in protein folding and processing between the plant and microorganism. Expression of the human proteins in plants has an advantage over expression of the human proteins in human or animal cells in that production of proteins from plants mitigates potential contamination of the protein fraction with human viruses and other disease causative agents found in human or animal sources. The present invention recognizes the desirability of expressing the human proteins in rice plants.

Rice endosperm contains several organelles devoted to the storage of nutrients used during seed germination and early seedling growth. These organelles include two different types of protein bodies, i.e. protein body I and protein body II, the starch granule, which comprises the majority of the endosperm components, and other minor structures. In rice endosperm, there are four main storage proteins, which are glutelin, prolamin, albumin and globulin. Prolamin is stored primarily in protein body I, and glutelin and globulin are primarily stored in protein body II. However, the storage location of albumin has not been conclusively determined.

There is a potential to increase recombinant protein expression by targeting recombinant proteins to different organelles, i.e. protein body I, protein body II or starch granules, in rice. Prior to the present invention, a recombinant protein has not been specifically targeted to protein body I or the starch granule in rice, although human proteins have been produced in dicot and monocot plants, for example, as disclosed in the references described below.

U.S. Pat. Nos. 6,417,429, 5,959,177, 5,639,947 and 5,202,422, all related patents, disclose the production of antibody molecules in transgenic tobacco plant leaves.

U.S. Pat. No. 5,767,363 discloses the use of a seed-specific promoter derived from ACP of Brassica napus, to affect and vary the expression of seed oils in rape and tobacco plants.

U.S. Pat. No. 6,303,341 discloses the production of immunoglobulins containing protection proteins in tobacco plant leaves, stems, flowers and roots.

U.S. Pat. No. 6,344,600 discloses the production of hemoglobin and myoglobin in plants. Example XI discloses expression of hemoglobin in maize seeds under the control of a rice actin promoter.

U.S. Pat. No. 6,569,831 discloses expression of human lactoferrin in plants utilizing plant protein promoters and signal peptides for intracellular targeting in plant cells.

U.S. Patent Application Publication No. 2002/0174453 discloses the production of antibodies in the plastids of tobacco plants.

U.S. Patent Application Publication No. 2002/0046418 discloses a controlled environment agriculture bioreactor for the commercial production of heterologous proteins in transgenic plants, particularly in the leaves of potato, tobacco and alfalfa plants.

Zheng et al, “The Bean Seed Storage Protein Beta-Phaseolin Is Synthesized, Processed, and Accumulated in the Vacuolar Type II Protein Bodies of Transgenic Rice Endosperm”, (1995) Plant Physiol. 109: 777-786 discloses use of the rice glutelin promoter to express the native common bean protein in rice and have this dicot plant protein accumulating in type II protein bodies in rice.

Yang et al., “Expression and Localization of Human Lysozyme in the Endosperm of Transgenic Rice” (2003) Planta, 216(4): 597-603 describes expression in rice of human lysozyme under the control of rice regulatory sequences. Likewise, Hwang et al., “Analysis of the Rice Endosperm-Specific Globulin Promoter in Transformed Rice Cells”, (2002) Plant Cell Reports 20: 842-847 describes expression of heterologous proteins in rice plants under control of rice regulatory sequences.

None of these patents discloses the production of heterologous proteins in rice using a monocot non-seed-storage protein promoter and corresponding signal peptide to express the heterologous protein. It is particularly desirable to provide for the production of human proteins in high yield free from contaminating source agents for the obvious benefits.

### SUMMARY OF THE INVENTION

The present invention includes three methods of producing seeds that accumulate a heterologous protein, preferably a non-plant protein. The first method of the invention is a method of producing seeds of a monocot plant such as a rice plant that accumulate a heterologous protein, which method comprises the following steps:

(a) stably transforming a monocot plant cell with a chimeric gene to obtain a transformed monocot plant cell, the chimeric gene comprising
(i) a promoter from a monocot non seed-storage protein gene,
(ii) a first DNA sequence, operably linked to said promoter, encoding a monocot seed-specific signal peptide, preferably a monocot seed-specific N-terminal signal peptide, capable of targeting a linked polypeptide to an intracellular region within a monocot seed cell, and
(iii) a second DNA sequence, operably linked to said promoter and linked in translation frame with the first DNA sequence, encoding the heterologous protein, wherein the first DNA sequence and the second DNA sequence together encode a fusion protein comprising the signal peptide and heterologous protein;

(b) growing a monocot plant from the transformed monocot plant cell to produce seeds that express the heterologous protein; and

(c) harvesting the seeds from the monocot plant grown in step (b) to obtain the seeds that accumulate the heterologous protein.

The second method of the invention is a method of producing seeds of an angiosperm, preferably a monocot such as a rice plant, that accumulate a heterologous protein, preferably a non-plant protein, in at least two intracellular regions within a cell, preferably an endosperm cell, of the seeds of the angiosperm, which method comprises the steps of:

(a) stably co-transforming a cell of the angiosperm, preferably a monocot such as the rice plant, with at least two independent chimeric genes to obtain a transformed angiosperm cell, the first chimeric gene comprising
(i) a first promoter from an angiosperm protein gene, preferably a monocot protein gene, more preferably a monocot seed protein gene, even more preferably a monocot non seed-storage protein gene,
(ii) a first DNA sequence, operably linked to the promoter, encoding a first angiosperm seed-specific signal peptide, preferably a monocot seed-specific signal peptide, more preferably a monocot seed-specific N-terminal signal peptide, capable of targeting a polypeptide linked thereto to a first intracellular region within an angiosperm seed cell, preferably an angiosperm endosperm cell, and
(iii) a second DNA sequence, operably linked to said promoter and linked in translation frame with the first DNA sequence, encoding the heterologous protein, wherein the first and second DNA sequences together encode a fusion protein comprising the first angiosperm seed-specific signal peptide and the heterologous protein,
   the second chimeric gene comprising
(i) a second promoter from an angiosperm protein gene, preferably a monocot protein gene, more preferably a monocot seed protein gene, even more preferably a monocot seed-storage protein gene,
(ii) a third DNA sequence, operably linked to the promoter, encoding a second angiosperm seed-specific signal peptide, preferably a monocot seed-specific signal peptide, more preferably a monocot seed-specific N-terminal signal peptide, capable of targeting a polypeptide linked thereto to a second intracellular region within an angiosperm seed cell, preferably an angiosperm endosperm cell, and
(iii) a fourth DNA sequence, operably linked to said promoter and linked in translation frame with the third DNA sequence, encoding the heterologous protein, wherein the third and fourth DNA sequences together encode a fusion protein comprising the second angiosperm seed-specific signal peptide and the heterologous protein,
wherein the first and second promoter are different, the first and

second angiosperm seed-specific signal peptides are different, and the first and second intracellular regions are different;

(b) growing an angiosperm plant from the transformed angiosperm cell to produce seeds that express the heterologous protein in at least two different intracellular regions; and

(c) harvesting the seeds from the angiosperm plant grown in step (b) to obtain the seeds of the angiosperm that accumulate the heterologous protein.

The third method of the invention is a method of producing seeds of an angiosperm, preferably a monocot such as a rice plant, that accumulate a heterologous protein, preferably a non-plant protein, in at least two different intracellular regions within a cell, preferably an endosperm cell, of the seeds of the angiosperm, which method comprises the steps of:

(a) stably transforming a first cell of the angiosperm, preferably the monocot such as the rice plant, with a first chimeric gene to produce a first transformed cell of the angiosperm, the first chimeric gene comprising
(i) a first promoter from an angiosperm protein gene, preferably a monocot protein gene, more preferably a monocot seed protein gene, even more preferably a monocot non seed-storage protein gene,
(ii) a first DNA sequence, operably linked to the promoter of (a)(i), encoding a first angiosperm seed-specific signal peptide, preferably a monocot seed-specific signal peptide, more preferably a monocot seed-specific N-terminal signal peptide, capable of targeting a polypeptide linked thereto to a first intracellular region within an angiosperm seed cell, preferably an angiosperm endosperm cell, and
(iii) a second DNA sequence, operably linked to said promoter and linked in translation frame with the first DNA sequence of (a)(ii), encoding the heterologous protein, wherein the first and second DNA sequences together encode a fusion protein comprising the first angiosperm seed-specific signal peptide and the heterologous protein;

(b) stably transforming a second cell of the angiosperm, preferably the monocot such as the rice plant, with a second chimeric gene to produce a transformed second cell of the angiosperm, the second chimeric gene comprising
(i) a second promoter from an angiosperm protein gene, preferably a monocot protein gene, more preferably a monocot seed protein gene, even more preferably a monocot seed-storage protein gene,
(ii) a third DNA sequence, operably linked to the promoter of (b)(i), encoding a second angiosperm seed-specific signal peptide, preferably a monocot seed-specific signal peptide, more preferably a monocot seed-specific N-terminal signal peptide, capable of targeting a polypeptide linked thereto to a second intracellular region within an angiosperm seed cell, preferably an angiosperm endosperm cell, and
(iii) a fourth DNA sequence, operably linked to said promoter and linked in translation frame with the third DNA sequence of (b)(ii), encoding the heterologous protein, wherein the third and fourth DNA sequences together encode a fusion protein comprising the second angiosperm seed-specific signal peptide and the heterologous protein,
wherein the first and second promoter are different, the first and second angiosperm seed-specific signal peptides are different, and the first and second intracellular regions are different;

(c) growing an angiosperm plant from the first transformed cell of (a) to produce a first angiosperm plant that express the heterologous protein in the first intracellular region;

(d) growing an angiosperm plant from the second transformed cell of (b) to produce a second angiosperm plant that express the heterologous protein in the second intracellular region;

(e) crossing the first and second angiosperm plants to produce a hybrid plant;

(f) growing the hybrid plant to produce seeds that express the heterologous protein in the first and second intracellular regions in the same seed cell; and

(g) harvesting the seeds from the hybrid plant to obtain the seeds of the angiosperm that accumulate the heterologous protein.

Another object of the invention is directed toward seeds produced by the first, second or third method of the invention described above.

### BRIEF DESCRIPTION OF THE DRAWINGS

FIG. 1 schematically shows the plasmid structures of three expression cassettes. The top expression cassette is plasmid pAPI302 containing a wheat puroindoline b (Tapur) promoter, signal-peptide sequence encoding a Tapur signal peptide, stuffer sequence and nopaline synthase (NOS) terminator. The middle expression cassette is plasmid pAPI308 prepared from pAPI302 by replacing the stuffer sequence with a codon-optimized human lysozyme gene fused in translational reading frame to the Tapur signal peptide. The bottom expression cassette is plasmid pAPI291 containing a Gns9 promoter, bar gene and NOS terminator.

FIG. 2 shows the results of a Western blot of human lysozyme expressed in transgenic rice grain extracts. Fifteen μl of grain extracts from TP309 and transgenic lines were loaded and separated in a 4-20% PAGE gel, followed by immuno-blotting with antiserum against human lysozyme. Lane 1: Molecular mass marker. Lane 2: Non-transgenic Taipei 309 (negative control). Lane 3: 0.3 μg purified human lysozyme (positive control). Lanes 4 and 5: Transgenic lines 308-73 and 159-53-1-16-2-18, respectively.

FIG. 3 presents Southern blot results of genomic DNA from two transgenic lines through 3 generations. Ten μg genomic DNA from transgenic plants was digested by XbaI and EcoRI and blotted onto a nylon membrane. The blots were probed for the human lysozyme gene. Lane 1: λDNA/HindIII DNA marker; lane 2: R₀ of 308-73; lanes 3, 5, and 7: R₁, R₂ and R₃ of transgenic line 308-73-6, respectively; lanes 4, 6, and 8: R₁, R₂ and R₃ of transgenic line 308-73-9, respectively; lane 9: Non-transgenic TP309; lane 10: 1× copy number equivalent of entire Tapur-Lys expression cassette digested by DraI and XhoI restriction enzymes. The 1,132 bp positive control band encompassing the entire chimeric gene is also shown in lane 10.

FIG. 4 shows an analysis of tissue-specific expression of lysozyme driven by the Tapur promoter from transgenic rice line 308-73-1-9-11. Thirty-five μl of total protein extracts from various tissues were loaded in 4-20% PAGE gels and immuno-blotted with antiserum against human lysozyme. Lane 1: Molecular mass marker. Lane 2: Root. Lane 3: Shoot. Lane 4: Stem. Lane 5: Leaf. Lane 6: Grain. Lane 7: Purified human lysozyme (positive control). Lane 8: Anther.

FIG. 5 shows the subcellular location of human lysozyme in rice endosperm. Rice glutelin was labeled with 10 nm diameter gold particles and human lysozyme was labeled with 6 nm diameter gold particles. PBI represents protein body I; PBII represents protein body II and S represents starch granule. FIG. 5(A) indicates that human lysozyme, labeled with the smaller particles, was localized in protein bodies I and II, and endogenous rice glutelin protein, labeled with the larger particles, was located predominantly in protein body II. In FIG. 5(B), human lysozyme was not located in the starch granule.

FIG. 6 shows the expression profile of human lysozyme during rice endosperm development in transgenic line 308-73-2. Ten spikelets were harvested at 7, 14, 21, 28, 35, 42 DAP and analyzed by a lysozyme activity assay.

### DETAILED DESCRIPTION OF THE INVENTION

Unless otherwise indicated, all terms used herein have the meanings given below or are generally consistent with the meanings that the terms have to those skilled in the art of the present invention. Practitioners are particularly directed to Sambrook et al. (1989) Molecular Cloning: A Laboratory Manual (Second Edition), Cold Spring Harbor Press, Plainview, N.Y., Ausubel FM et al. (1993) Current Protocols in Molecular Biology, John Wiley & Sons, New York, N.Y., and Gelvin et al., eds. (1990) Plant Molecular Biology Manual, for definitions and terms of the art.

As used herein, the phrase “non seed-storage protein” means a seed protein which is not a storage protein. In other words, a non seed-storage protein is a protein which is not mainly synthesized and accumulated during seed maturation, stored in the dry grain, and mobilized during maturation. Thus, the term “non seed-storage protein” excludes rice albumin, arachin, avenin, cocosin, conarchin, concocosin, conglutin, conglycinin, convicine, crambin, cruciferin, cucurbitin, edestin, excelesin, gliadin, rice globulin, rice glutelin, gluten, glytenin, glycinin, helianthin, barley hordein, kafirin, legumin, napin, oryzin, pennisetin, phaseolin, rice prolamin, psophocarpin, secalin, vicilin, vicine and zein. Examples of non seed-storage proteins include, but are not limited to, puroindoline b, protein disulfide isomerase (PDI), rice heat shock 70 (BIP) proteins and actin.

“Heterologous protein” is a protein originally encoded by a DNA sequence exogenous to the host plant. Preferably, “heterologous protein” is a protein originally encoded by a non-plant DNA sequence.

As used herein, the word “promoter” means a transcription promoter recognizable by the transcription machinery of the angiosperm cell. Examples of the promoter are rice glutelin-1 (Gt1) promoter, rice actin promoter, promoter 35S (35S) or double constitutive promoter (d35S) of cauliflower mosaic virus, promoters PGA1 and PGA6 of Arabidopsis thaliana, maize y-zein promoter, barley high-molecular weight glutenin promoter, promoter PCRU of the radish cruciferin gene and chimeric promoter super-promoter PSP of Agrobacterium tumefaciens. The promoter preferably is a promoter from (a) puroindoline protein, preferably from wheat, (b) protein disulfide isomerase gene, or (c) heat shock 70 (BIP) gene.

When a first DNA sequence is “operably linked” to a promoter and a second DNA sequence is “linked in translation frame” with the first DNA sequence, it means that, preferably, the 3′ end of the promoter is linked to the 5′ end of the first DNA sequence, and the 3′ end of the first DNA sequence is linked to the 5′ end of the second DNA sequence, so that the promoter controls the transcription of both the first and second DNA sequences and the translation of the chimeric gene, preferably, results in a fusion protein having the carboxy terminal of a signal peptide linked to the amino terminal of a heterologous protein. Alternatively, the 3′ end of the promoter is linked to the 5′ end of the second DNA sequence, and the 3′ end of the second DNA sequence is linked to the 5′ end of the first DNA sequence, and the promoter controls the transcription of both the second and first DNA sequences.

The 3′ end of the chimeric gene may contain 3′ regulatory sequences such as a transcription terminator recognizable by the transcriptional machinery of the angiosperm cell. Examples of plant-derived transcription terminator sequences are the nos polyA terminator of the nopaline strain of Agrobacterium tumefaciens and the polyA terminators for the 35S and 19S transcripts of cauliflower mosaic virus.

The term “blood protein” refers to one or more proteins, or biologically active fragments thereof, found in normal human blood, including, without limitation, hemoglobin, alpha-1-antitrypsin, fibrinogen, human serum albumin, prothrombin/thrombin, antibodies, blood coagulation factors (ie; Factor V, Factor VI, Factor VII, Factor VIII, Factor IX, Factor X, Factor XI, Factor XII, Factor XIII, Fletcher Factor, Fitzgerald Factor and von Willebrand Factor), and biologically active fragments thereof.

The term “milk protein” refers to one or more proteins, or biologically active fragments thereof, found in normal human milk, including lactoferrin, lysozyme, alpha-1 anti-trypsin, antibodies, protein factors, immune molecules, and biologically active fragments thereof.

“Seed maturation” refers to the period starting with fertilization in which metabolizable reserves, e.g., sugars, oligosaccharides, starch, phenolics, amino acids, and proteins, are deposited, with and without vacuole targeting, to various tissues in the seed (grain), e.g., endosperm, testa, aleurone layer, and scutellar epithelium, leading to grain enlargement, grain filling, and ending with grain desiccation.

In the first method of the invention for producing monocot seeds, such as rice seeds, that accumulate a heterologous protein, the promoter from the monocot non seed-storage protein in the chimeric gene preferably corresponds to the seed-specific signal peptide encoded by that gene. The monocot seed cell preferably is a monocot endosperm cell, more preferably a rice endosperm cell.

In the second and third methods of the invention for producing seeds of an angiosperm that accumulate a heterologous protein, the promoter of the angiosperm protein gene is preferably a promoter taken from a gene encoding the angiosperm seed-specific signal peptide encoded by the first or third DNA sequence in the same chimeric gene. Therefore, in the second or third method of the invention, the first promoter is preferably from a gene encoding the first angiosperm seed-specific signal peptide, and the second promoter is preferably from a gene encoding the second angiosperm seed-specific signal peptide.

The intracellular region within a monocot seed cell (in the first method of the invention) or an angiosperm seed cell (in the second or third method of the invention) targeted by the signal peptide can be an intracellular compartment, e.g. an organelle such as a vacuole, protein body, starch granule, peroxisome, endoplasmic reticulum, Golgi complex, mitochondria and chloroplast, inside the cell wall of the seed cell, which preferably is an endosperm cell.

A “signal sequence” is a DNA sequence encoding a signal peptide. A “seed-specific signal peptide” is a peptide that preferentially targets a linked polypeptide to an intracellular region of a seed cell. The signal peptide can be a C-terminal signal peptide or, preferably, an N-terminal signal peptide. When an N-terminal signal peptide is used, the carboxy terminal amino acid of the N-terminal signal peptide joins the amino terminal amino acid of the linked polypeptide. Examples of the N-terminal signal peptide are wheat puroindoline b signal peptide, the rice globulin signal peptide (Glb) and the rice glutelin-1 (Gt1) signal peptide. When a C-terminal signal peptide is used, the amino terminal amino acid of the C-terminal signal peptide joins the carboxy terminal amino acid of the linked polypeptide. An example of the C-terminal signal peptide is barley lectin carboxy terminal propeptide. Preferably, according to the invention, the signal peptide targets the linked polypeptide to a region such as an organelle of the cell of the angiosperm or monocot such as rice.

The invention can optimize the expression of heterologous proteins in rice in at least one of two ways. Monocot seed-storage protein promoters and seed-specific signal sequences, preferably seed-specific signal sequences corresponding to the monocot non seed-storage protein promoters, are used to express heterologous proteins such as human proteins in rice. Additionally, a chimeric gene containing a monocot seed-storage protein promoter can be combined via co-transformation or gene stacking via a hybrid breeding approach to target at least two rice organelles to attain expression of even larger quantities of the target heterologous protein. This second expression cassette can comprise a monocot seed-storage protein promoter/signal sequence regulating expression in the rice seed, and targeting the heterologous protein to a different cellular compartment than targeting achieved by the first non seed-storage promoter/signal sequence expression cassette. An additive effect can be achieved by introducing another expression cassette into the rice plant, where the second cassette has a different targeting signal than the first. Also, two plants independently capable of expressing a heterologous gene of interest, can be crossed to form a hybrid plant that expresses both chimeric genes. The heterologous genes can be the same gene, thus optimizing expression of a single protein of interest by directing accumulation of this gene in two different organelles in the host plant cell endosperm cell.

Accordingly, the invention includes a method of producing rice seeds that accumulate a target heterologous protein, preferably a non-plant protein (e.g. an animal protein, further by example, a human protein), at high level. This level can be as high as 200 μg of a non-plant protein expressed per individual rice seed In order to achieve this expression, a rice plant cell is stably transformed with a chimeric gene. Stable transformation means that the plant cell has a non-native (heterologous) nucleic acid sequence, preferably, integrated into its nucleic acid, such as genome, that is maintained through two or more generations. A host cell is a cell containing a vector and supporting the replication and/or transcription and/or expression of the heterologous nucleic acid sequence. Preferably, according to the invention, the host cell is a rice plant cell. Other host cells (i.e, bacterial) may be used as secondary hosts to move DNA to a desired plant host cell. A plant cell refers to any cell derived from a plant, including undifferentiated tissue (e.g., callus) as well as plant seeds, pollen, progagules, embryos, suspension cultures, meristematic regions, leaves, roots, shoots, gametophytes, sporophytes and microspores.

The chimeric gene can preferably comprise a promoter/signal peptide combination from a monocot non seed-storage protein. For example, a promoter from a non seed-storage protein gene normally expressed in wheat, barley or other monocots can be used. In an exemplary fashion, this invention provides expression in rice under regulatory control of a wheat puroindoline b promoter. The wheat puroindoline protein is normally targeted by the puroindoline signal peptide to the surface of the wheat endosperm starch granule (Rahman et al “Cloning of a wheat 15 kDa grain softness protein (GSP) is a mixture of different purindoline-like polypeptides”, (1994) Eur. J. Biochem. 223: 917-925). Unexpectedly, expression in rice of a heterologous protein under control of the wheat puroindoline gene promoter and puroindoline signal peptide, targets the heterologous protein to the rice protein body II organelle instead of the rice starch granule. Similar results can be achieved when the expression in rice of a heterologous protein is under control of one of the following combinations: rice actin gene promoter/signal peptide for rice actin, disulfide isomerase gene promoter/signal peptide for disulfide isomerase gene, and BIP gene promoter/signal peptide for BIP gene. Various combinations of these promoters and signal peptides are also contemplated in accordance with the invention.

Generally, expression vectors for use in the present invention are chimeric nucleic acid constructs (or expression vectors or cassettes), designed for expression in plants containing associated upstream and downstream sequences, including the promoters and signal peptides mentioned above.

The vector will also comprise a second DNA sequence, linked in translation frame with the first DNA sequence, encoding a heterologous protein, preferably a non-plant protein such as a animal protein, e.g. a mammalian protein, with a human protein more preferred. The first DNA sequence and the second DNA sequence together encode a fusion protein comprising a signal peptide and the heterologous protein. The second DNA sequence can encode any heterologous protein, e.g. an animal or human protein, that it is desirable to be produced in the plant system. For example, the second DNA sequence can encode a human protein selected from the group consisting of a human blood protein, human milk protein, human growth factor, human gastrointestinal delivered peptide, human protein required for cell culture, lipase, amylase, colony stimulating factor, cytokine, interleukin, integrin, T cell receptor, immunoglobulin, growth factor, growth hormone, a vaccine, lysozyme, lactoferrin, lactoperoxidase, kappa-casein, hemoglobin, alpha-1-antitrypsin, fibrinogen, antithrombin III, human serum albumin, trypsinogen, aprotinin, transferrin, human growth hormone, an antibody, insulin, insulin-like growth factor, epithelial growth factor, intestinal trefoil factor, granulocyte colony-stimulating factor (G-CSF), and macrophage colony-stimulating factor (M-CSF).

The animal and human proteins produced in accordance with the invention also include all variants thereof, whether allelic variants or synthetic variants. A “variant” human blood protein-encoding nucleic acid sequence may encode a variant human blood protein amino acid sequence that is altered by one or more amino acids from the native blood protein sequence, preferably at least one amino acid substitution, deletion or insertion. The nucleic acid substitution, insertion or deletion leading to the variant may occur at any residue within the sequence, as long as the encoded amino acid sequence maintains substantially the same biological activity of the native human blood protein. In another embodiment, the variant human blood protein nucleic acid sequence may encode the same polypeptide as the native sequence but, due to the degeneracy of the genetic code, the variant has a nucleic acid sequence altered by one or more bases from the native polynucleotide sequence.

The variant nucleic acid sequence may encode a variant amino acid sequence that contains a “conservative” substitution, wherein the substituted amino acid has structural or chemical properties similar to the amino acid which it replaces and physicochemical amino acid side chain properties and high substitution frequencies in homologous proteins found in nature (as determined, e.g., by a standard Dayhoff frequency exchange matrix or BLOSUM matrix). Standard substitution classes include six classes of amino acids based on common side chain properties and highest frequency of substitution in homologous proteins in nature, as is generally known to those of skill in the art and may be employed to develop variant human blood protein-encoding nucleic acid sequences.

The rice plant, suitably transformed with the chimeric gene(s) of interest can then be grown from the transformed rice plant cell for a time sufficient to produce seeds containing the heterologous protein. The seeds are then harvested from the plant. Formation of the transgenic seeds, including transformation and expression of the gene of interest, growth of the plants, and harvesting of the protein enriched seeds is described in U.S. patent application Ser. Nos. 10/411,395 and 10/377,381, which are incorporated by reference in their entirety.

The promoter regulating expression of a heterologous target gene in rice can be obtained from a monocot non seed-storage protein gene. For example, a promoter of a gene from a monocot other than rice can be employed. Thus, for example, the promoter can be from a gene selected from the group consisting of a protein from wheat, rye, barley, sorghum, tricale, and other monocots. The first method of the invention is exemplified herein using a promoter/signal sequence of a wheat puroindoline b protein, but expression can also be accomplished, for example, with any monocot non seed-storage protein promoter, for example a promoter from the protein disulfide isomerase (PDI) gene (Ciaffi et al, “Molecular characterization of gene sequences coding for protein disulfide isomerase (PDI) in durham wheat (Triticum turgidum spp durham)” (2001), Gene 265: 147-56) or heat shock 70 (BIP) gene (Li et al, “Rice prolamine protein body biogenesis: a BiP-mediated process” (1993) Science 262: 1054-56). Purification of the non-plant protein from the harvested seeds can be accomplished by standard methods, see for example U.S. patent application Ser. No. 10/411,395. For instance, the purification can be accomplished by processing the harvested seeds to obtain a fraction enriched for proteins, and isolating the non-plant protein from the enriched fraction by methods known in the art.

The invention further contemplates rice seeds containing a heterologous protein, preferably a non-plant protein, produced by one of the methods disclosed herein. The rice seeds produced contain the heterologous protein that has been expressed, preferably, in a particular organelle by targeting expression to that organelle using, preferably, a monocot non seed-storage promoter such as the promoter from the puroindoline gene, protein disulfide isomerase gene, heat shock 70 (BIP) gene or actin gene, and a monocot seed-specific signal peptide. More preferably, the promoter is taken from a gene encoding the signal peptide.

Expression vectors used in the invention can include the following operably linked components that constitute a chimeric gene: a promoter from the gene of a monocot non seed-storage protein, e.g. wheat puroindoline, a first DNA sequence, preferably a wheat puroindoline signal sequence, operably linked to the promoter, encoding a signal peptide such as an N-terminal leader peptide or a C-terminal signal peptide, and a second DNA sequence, linked in translation frame with the first DNA sequence, encoding a heterologous protein, e.g. an animal or human protein. The first and second DNA sequences can be linked in either order.

The chimeric gene, in turn, can typically be placed in a suitable plant-transformation vector having (i) companion sequences upstream and/or downstream of the chimeric gene which are of plasmid or viral origin and provide necessary characteristics to the vector to permit the vector to move DNA from bacteria to the desired plant host; (ii) a selectable marker sequence; and (iii) a transcriptional termination region generally at the opposite end of the vector from the transcription initiation regulatory region.

Numerous types of appropriate expression vectors, and suitable regulatory sequences are known in the art for a variety of plant host cells. The promoter region can be regulated in a manner allowing for expression under seed-maturation conditions. In one aspect of this embodiment of the invention, the expression construct includes a promoter, e.g. wheat puroindoline b promoter, from a monocot non seed-storage protein gene. Promoters for use in the invention can be typically derived from wheat purindolines or other monocot plants as directed for a particular construct.

The invention also includes expressing target heterologous proteins in a rice seed where more than one cassette is used and the protein(s) in each cassette is targeted to different organelles in the rice seed. Accordingly, there is provided a method of producing monocot seeds that accumulate a selected heterologous protein to at least two different intracellular region, e.g. two organelles, of a host seed comprising the steps of stably co-transforming a rice plant cell with at least two chimeric genes each comprising different promoters that target the expressed protein to a different organelle in the rice seed. Each promoter comprises a promoter from a monocot gene, and a DNA sequence, operably linked to the promoter, encoding a monocot plant seed-specific signal peptide capable of targeting a polypeptide linked thereto to a rice seed endosperm cell. A second DNA sequence, linked in translation frame with the first DNA sequence, encoding a non-plant protein, is also included. The first DNA sequence and the second DNA sequence together encode a fusion protein comprising an N-terminal or C-terminal signal peptide and the non-plant protein. The rice plant is grown from the transformed rice plant cell for a time sufficient to produce rice seeds containing quantities of non-plant protein expressed in at least two different organelles. The rice seeds are harvested from the plant. The construction of the two or more chimeric gene cassettes, co-transformation, growth and harvesting can be accomplished as described earlier herein, with the simple change that two or more genes are expressed and each of the genes targets the heterologous protein to a different organelle in the rice endosperm cell. Accordingly, and in order to achieve this effect, each chimeric gene will be under the regulatory control of a different promoter. For instance, one chimeric gene can be under the regulatory control of a monocot seed-storage protein and another chimeric gene can be under the regulatory control of a monocot non seed-storage protein. Preferably, in each of the chimeric genes, the promoter and the signal peptide are derived from the seed-storage or non seed-storage protein. Optimization of the system can be achieved using a rice promoter/signal peptide of a seed storage protein in one cassette, e.g. a Gt1 promoter/Gt1 signal peptide, and a monocot non seed-storage protein promoter in the other, e.g. a promoter of the wheat purindoline b gene as described in the examples. Signal sequences optionally can be selected to correspond to the same gene as the promoter.

There are a number of possible ways to obtain plant cells containing more than one expression construct. In one approach, plant cells are co-transformed with a first and second construct by inclusion of both chimeric genes in a single transformation vector or by using separate vectors, each of which expresses the desired gene. The second construct can be introduced into a plant that has already been transformed the first chimeric gene construct, or alternatively, transformed plants, one having the first construct and one having the second construct, can be crossed to bring the constructs together in the same plant.

To be used in the second or third method of the invention, the two or more cassettes can comprise, for example, a monocot seed storage protein promoter and a monocot non seed-storage protein promoter. As described earlier, the invention can include purifying the non-plant protein from the harvested seeds, and retrieving the selected protein from the harvested seeds by processing the seeds to obtain a fraction enriched for protein, and isolating the non-plant protein from the enriched fraction. The invention includes a seed produced by the method of co-transformation of more than one chimeric gene expression systems as described herein, and an isolated non-plant protein produced by the same methods. As listed earlier, the heterologous proteins expressed in a co-transformation system can include any human proteins desirable to be produced in plants, particularly rice seeds.

Additional aspects of the invention include an expression system with two or more chimeric genes targeting expression to two or more intracellular regions, e.g. organelles, within the rice endosperm cell wherein the system is constructed by obtaining two or more independent rice transformants and crossing the seeds of selected transformants to produce a hybrid plant that can express all the chimeric genes, targeted to two or more intracellular regions.

Exemplification of the invention includes use of targeting signals obtained from a monocot non seed-storage protein gene e.g. wheat grain, specifically a promoter/signal peptide of puroindoline b that is normally deposited on the surface of the wheat starch granule (Rahman et al, “Cloning of a wheat 15 kDa grain softness protein (GSP) is a mixture of different purindoline-like polypeptides”, (1994) Eur. J. Biochem. 223: 917-925). Puroindoline b protein is a basic cysteine-rich protein expressed in wheat grain affecting grain softness (Krishnamurthy et al., “Expression of wheat puroindoline genes in fransgenic rice enhances grain softness”, (2001) Nat. Biotechnol., 19(2): 162-6). The tissue expression pattern of the puroindoline b promoter in transgenic rice grains shows endosperm-specific expression in rice grain (Digeon et al., “Cloning of a wheat puroindoline gene promoter by IPCR and analysis of promoter regions required for tissue-specific expression in transgenic rice seeds”, (1999) Plant Mol. Biol., 39(6): 1101-1112) and grain softness and resistance to fungal diseases are enhanced when an intact wheat puroindoline b gene is introduced into rice plants. The invention described herein is exemplified by showing that a human lysozyme gene under the control of the puroindoline b (Tapur) promoter and Tapur signal peptide results in lysozyme accumulation predominantly within protein body I in transgenic rice seeds, with the potential for additive effects when used in conjunction with a Gt1 promoter/signal peptide expression cassette which targets heterologous lysozyme protein expression to protein body II. The methods of the invention can use the Tapur promoter and signal peptide to express human lysozyme in rice seeds optimized by independently expressing the gene of interest (lysozyme) in conjunction with the Gt1 expression cassette as described in Huang et al. (“Expression of functional recombinant human lysozyme in transgenic rice cell culture”, (2002) Transgenic Res. 11(3): p. 229-39).

According to the present invention, wheat puroindoline b promoter and signal peptide can be used to direct the expression of human proteins in rice grains. The Tapur signal peptide is properly cleaved by rice endosperm cells during protein maturation. Human lysozyme expression driven by the Tapur promoter is endosperm-specific and the transgene is genetically stable through multiple generations. Electron microscopy results demonstrated that human lysozyme protein was localized to protein bodies I and II under the control of the wheat Tapur promoter/signal peptide. An additive improvement in yield for lysozyme expression was obtained when combining the wheat Tapur and rice Gt1 expression cassettes respectively.

### EXAMPLE 1

### Construction of Plasmids

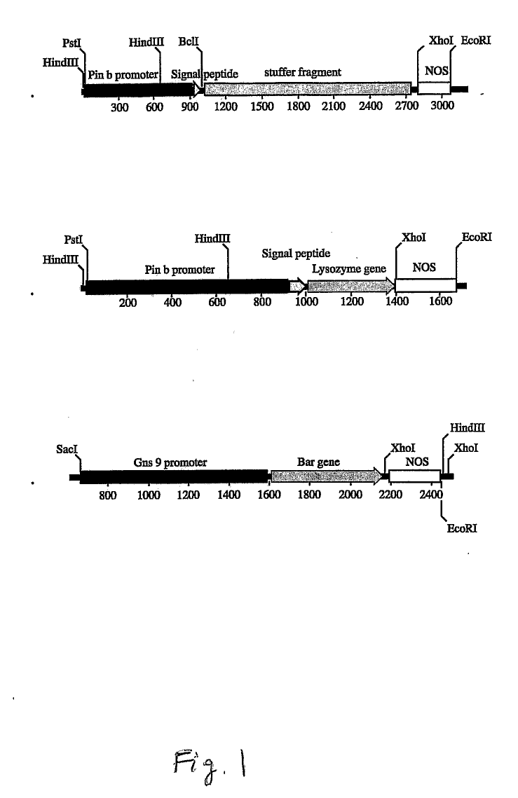

A 1,061 bp fragment containing the wheat puroindoline b promoter and signal peptide was amplified from genomic DNA of Triticum aesvestium, cv. Bobwhite by Pfu DNA polymerase using reverse primer: 5′-GGGAATATTGTACCAGCCGCCAACTTCTGA-3′ and forward primer: 5′-CCGCTGCAGCTCCAACATCTTATCGCAACATCC-3′, designed from the sequences of Genbank accession number AJ000548. The reverse primer introduces a silent mutation into the signal peptide, creating a Bcl I site for in-frame fusion of a recombinant gene. The fragment was cloned into the pCR2.1 vector (Invitrogen, Carlsbad, Calif.). After confirmation by sequencing analysis, the fragment was cut by SphI, and cloned into the NaeI/SphI site of API241 (Hwang et al., “Analysis of the rice endosperm-specific globulin promoter in transformed rice cells”, (2002) Plant Cell Report 20: 842-847). This backbone contains a 1.8 kb stuffer fragment, the nopaline synthase terminator (NOS), and an ampicillin resistance selectable marker gene. This intermediate construct was designated API302 (FIG. 1, top). Next, API302 was cut with Bcl I, blunted by Mung Bean Nuclease, and then digested with XhoI to remove the stuffer fragment. A human lysozyme gene (GenBank accession No. X63990), codon-optimized with rice preferred codons (Operon Technologies, Alameda, Calif.), was inserted into the vector in place of the stuffer fragment. The resulting construct was designated as pAPI308 (FIG. 1, middle).

For pAPI291 plasmid construction, a 871 bp fragment containing the phosphinothrin acetyltransferase gene (Bar) and NOS was obtained by digestion of pJH2600 with PstI blunted by T4 DNA polymerase, then digested by EcoRI, and then cloned into pAPI76 digested by XbaI and blunted by T4 DNA polymerase, followed by digestion with EcoRI. The resulting plasmid was designated as pAPI291 (FIG. 1, bottom).

### Example 2

### Generation of Transgenic Rice Plants

A selectable marker construct pAPI146, consisting of the hygromycin B phosphotransferase (Hph) gene driven by the Gns9 promoter and followed by the NOS terminator (Huang et al., “The tissue-specific activity of a rice beta-glucanase promoter (Gns9) is used to select rice transformants”, (2001) Plant Sci. 61: 589-595)), was used as the selectable marker in all transformations except for the gene stacking experiment. For gene stacking, the calli derived from a transgenic line, 159-53, already carrying pAPI146, so a second selectable marker construct, pAPI291 carrying the Gns9 promoter, Bar, and NOS terminator was used for selection of transgenic calli. Microprojectile-mediated transformation of rice was carried out according to the procedure described in Yang et al. (“Expression of the REB transcriptional activator in rice grains improves the yield of recombinant proteins whose genes are controlled by a Reb-responsive promoter”, (2001) Proc Natl Acad Sci USA, 98(20): 11438-43).

### Lysozyme Activity Assay

Soluble protein extracts were prepared by grinding ten pooled R1 seeds from each R0 transgenic plant in 10 ml of chilled extraction buffer (PBS pH 7.4 plus 0.35 M NaCl). Suspensions were rocked gently at 4° C. for 24 hours, followed by centrifugation at 14,000 rpm in a microcentrifuge for 10 minutes at 4° C. Lysozyme activity was assayed as described in Yang et al. (“Expression of the REB transcriptional activator in rice grains improves the yield of recombinant proteins whose genes are controlled by a Reb-responsive promoter”, (2001) Proc Natl Acad Sci USA 98(20): p. 11438-43).

### Lysozyme Expression Profile During Endosperm Development

Spikelets were harvested at 7, 14, 21, 28, 35, 42, and 49 days after pollination (DAP) and stored at −70° C. Total protein concentration of the extracts was determined using the Bio-Rad Protein Assay system (BioRad, Hercules, Calif.). Lysozyme extracts and activity assays were performed as described above.

### EXAMPLE 3

### Isolating the Heterologous Protein

Total protein extracts of seeds and other tissues were prepared by grinding the tissue under liquid nitrogen, then adding protein extraction buffer (66 mM Tris, pH 6.8, 2% SDS, 2% β-mercaptoethanol). Proteins were separated by 4-20% polyacrylamide gel electrophoresis (PAGE), and then transferred to nitrocellulose membranes according to the manufacturer's instructions (BioRad). Blots were blocked in blocking solution (PBS, pH 7.4+5% non-fat dried milk, 0.02% sodium azide, 0.05% Tween 20) at 4° C. overnight. Next, the blot was incubated with a 1:2500 dilution of anti-lysozyme antibody (CalBiochem, San Diego, Calif.) in blocking solution for 1 hour at room temperature. Blots were washed three times with PBS, and then incubated with a 1:4000 dilution of AP-conjugated rabbit anti-sheep IgG antibody (Sigma, St. Louis, Mo.) in blocking solution for 1 hour at room temperature. Finally, the blots were washed 3 times with TBS (pH 7.4) and developed with 5-bromo-4-chloro-3-indoyl phosphate-nitroblue tetrazolium (Sigma).

### N-Terminal Sequencing

Rice protein extracts were separated by 10-20% SDS-PAGE followed by electroblotting to a PVDF membrane (Bio-Rad). The membrane was then stained with 0.1% Coomassie Brilliant Blue R-250 in 40% methanol and 1% glacial acetic acid for 1 minute. Destaining was conducted with 50% methanol with several changes until the desired background was obtained. The blot was thoroughly washed with H₂O and the human lysozyme band was cut out and subjected to N-terminal sequencing by Edman chemistry at the Molecular Structure Facility of University of California, Davis.

### Southern Blot Analysis

Genomic DNA was isolated from generations of transgenic plants (R₀-R₃) as described in Dellaporta et al. (“A plant DNA mini preparation: version II”, (1983) Plant Mol. Biol. Report, 1: 19-21). About five μg of the rice genomic DNA was digested by XbaI and EcoRI and then blotted onto a Nylon membrane according to manufacturer's instructions. Blot was probed with the lysozyme gene.

### Transmission Electron Microscopy

Immature endosperm was harvested at 14 DAP. The fixation and slice preparation followed the procedure described in Yang et al. (“Expression and localization of human lysozyme in the endosperm of transgenic rice”, (2003) Planta, 216(4): 597-603). For detection of recombinant human lysozyme and the native rice storage protein glutelin, an antiserum against human lysozyme from sheep and an antiserum against glutelin from rabbits was incubated with section at RT for 1 hr, followed by PBS washing, and then incubated with the secondary antiserum against sheep IgG which conjugated with 6 nm gold particles and antiserum against rabbits IgG conjugated with 10 nm gold particles, at RT for 1 hr. After PBS washing, sections were stained with 1% uranyl acetate and microscopic observation was carried out with transmission electron microscope JEM-100CX.

### EXAMPLE 4

### Generation of Transgenic Plants and Monitoring of the Lysozyme Expression Level

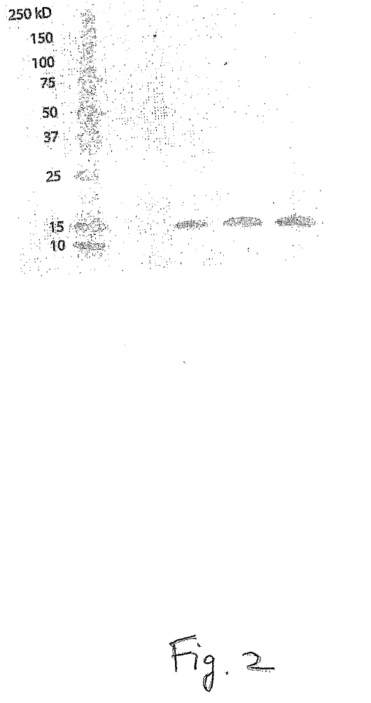

Plasmid pAPI308 carrying the Tapur promoter and signal peptide (FIG. 1, middle) for expression of the human lysozyme gene was co-transformed into rice variety Tapei 309 together with a selectable marker construct, pAPI146, via biolistic bombardment. A total of 318 transgenic plants were obtained. These plants were grown in a greenhouse until mature, i.e. fully differentiated, and mature seeds were harvested for analysis. From the 318 transgenic plants, 161 set of seeds were retrieved. For screening of lysozyme expression in R₁ seeds from R₀ plants, 10 R₁ seeds from each fertile transgenic plant were ground in 10 ml of extraction buffer (PBS, pH 7.4 0.35 M NaCl). The lysozyme amounts in the extracts were quantified by a turbidometric activity assay (Yang et al., “Expression and localization of human lysozyme in the endosperm of transgenic rice”, (2003) Planta, 216(4): 597-603). In lines with detectable lysozyme activity, the expression level in R₁ seeds ranged from 18.9 to 41.6 μg/grain with an average of 26.6±8.3 μg/grain (see Table 1). There was no significant difference between this value and the average expression level for R₁ seeds carrying the Gt1-Lys cassette, 28.4±19.9 μg/grain (P=0.65). Presence of lysozyme in these extracts was confirmed by specific reaction with an anti-lysozyme antibody on a Western blot (FIG. 2), indicating the same apparent molecular mass as purified native human lysozyme. To confirm whether the cleavage of the puroindoline b signal peptide from the mature lysozyme was correctly performed in rice grain, the N-terminal sequence of the recombinant lysozyme was determined to be identical to that of native human lysozyme (Table 2). This demonstrated the wheat puroindoline b signal peptide is properly processed in rice seed endosperm cells.

TABLE 1
Statistical analysis of human lysozyme expression level in R₁
seed detailing different expression strategies

| | | | | | 308/159 (t- |
|---|---|---|---|---|---|
| Approaches | Range (μg/grain) | Average ± S | 308 (t-Test) | 159 (t-Test) | Test |
| 308 | 18.9-41.63 | 26.57 ± 8.27 | | | |
| 159 | 15.63-71.93 | 28.72 ± 19.94 | 0.65 | | |
| 159/308 | 22.2-110 | 56.08 ± 28.14 | 0.004** | 0.0165* | |
| 308//159 | 58.4-201.5 | 136.99 ± 26.22 | 5.68 × 10⁻²⁴** | 6.36 × 10⁻¹²** | 3.53 × 10⁻⁸** |

Note:
*= P < 0.05;
**= P < 0.01

TABLE 2
N-terminal sequences comparison of rLys and
native human lysozyme

| | |
|---|---|
| Native human lysozyme | KVFERCELART |
| Rice recombinant human lysozyme | KVFER( )ELART |

Note: Cysteine can not be detected in amino acid sequencing reaction

### Genetic Stability of Transgenic Plants Through Multiple Generations

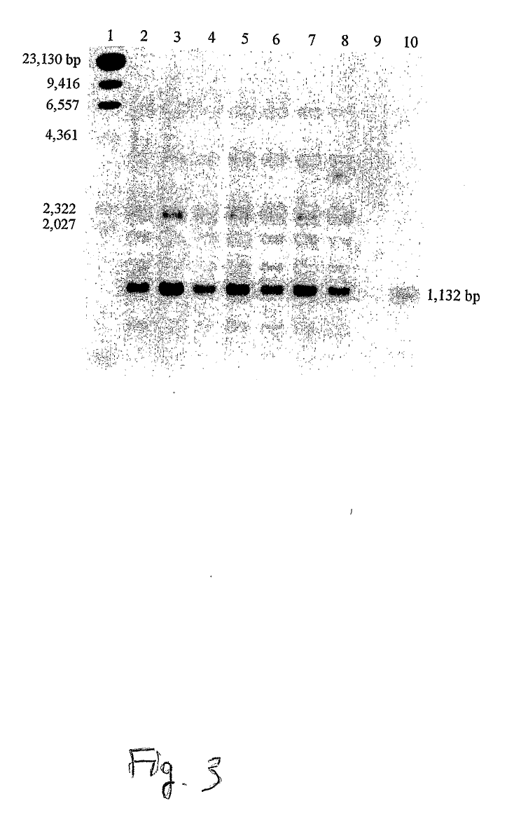

To determine the genetic stability of the transgene in the rice genome, Southern blot analysis of two transgenic lines from one event for generations R₀ to R₃ was performed. The banding patterns of the two lines were identical through 4 generations, demonstrating the stability of the transgene in these lines (FIG. 3). The results also showed that the transgene was present in the rice genome in multiple copies. The copy number was estimated to be 4-5 copies of the entire cassette, based on the intensity of bands equal in size to the complete cassette, plus at least 5 truncated copies. These bands exhibited different molecular masses, indicating the loss of one restriction enzyme site in the expression cassette.

### EXAMPLE 5

### Tissue Specificity and Subcellular Localization of Human Lysozyme in Rice Grain

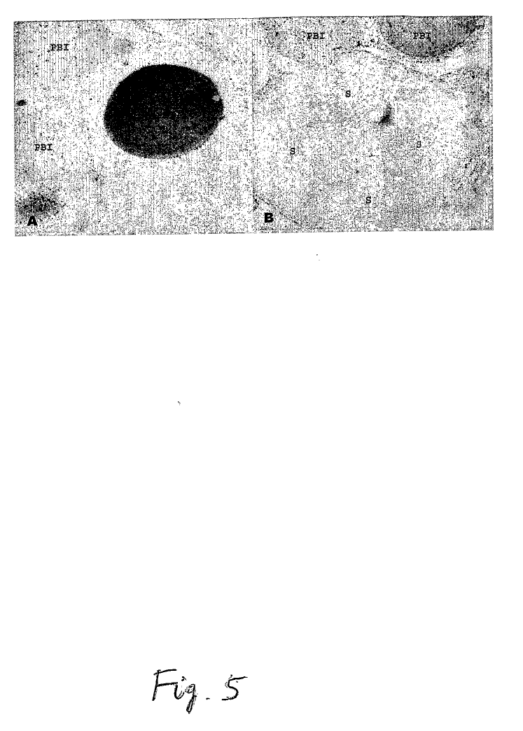

To determine the tissue specificity of the Tapur-lysozyme expression cassette in transgenic rice, total protein was extracted from the root, leaf, stem, anther and seeds of transgenic plants. These tissue extracts were tested for the presence of lysozyme by Western blot analysis. Lysozyme was detected only in seed endosperm, not in root, leaf, stem or anther (FIG. 4).

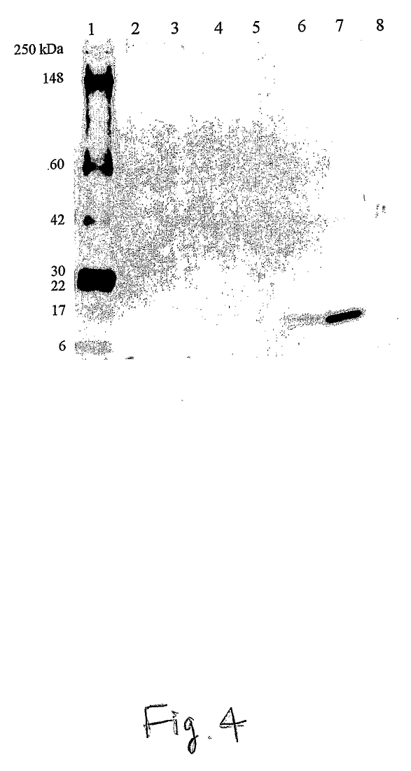

To determine the subcellular localization of human lysozyme expressed from the Tapur promoter in rice endosperm, 14 DAP immature endosperm tissue was harvested and studied using transmission electron microscopy. Surprisingly, no lysozyme was detected in or on the starch granule. Instead, human lysozyme was localized to both protein bodies I and II. Endogenous rice glutelin which was monitored as an internal control was predominantly localized to protein body II (FIG. 5). The results indicated that human lysozyme could be targeted to both protein bodies I and II in rice endosperm using the Tapur promoter cassette and Tapur signal peptide sequence, so the Tapur promoter and signal peptide can be used in a cell-compartment filling strategy (a heterologous protein can be targeted to different compartments of an angiosperm cell by selection of different promoters and signal peptides).

### EXAMPLE 6

### Expression Profile of Human Lysozyme during Rice Endosperm Development

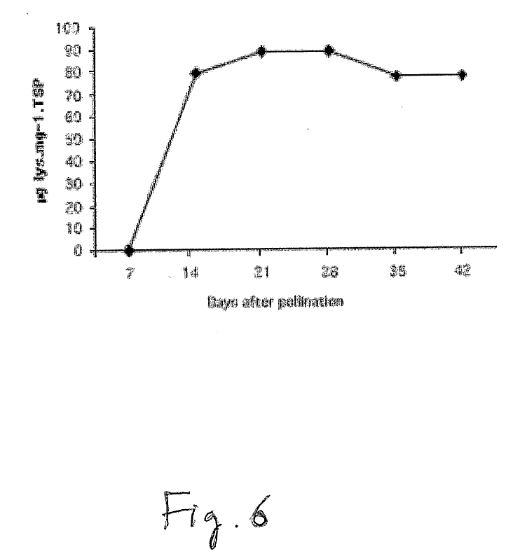

The expression profile of lysozyme in rice grain from transgenic line 308-73 was monitored at 7, 14, 21, 28, 35, and 42 DAP. Lysozyme content increased dramatically between 7 and 14 DAP, continued to increase through 21 DAP, then decreased slightly and plateaus at 35 DAP with a level of 78 μg·mg ⁻¹ total soluble protein through seed maturity (FIG. 6). This was similar to the human lysozyme expression profile when driven by the globulin promoter and signal peptide (Yang et al., “Expression and localization of human lysozyme in the endosperm of transgenic rice”, Planta, 2003. 216(4): p. 597-603). This profile conflicts with the results of Digeon et al (“Cloning of a wheat puroindoline gene promoter by IPCR and analysis of promoter regions required for tissue-specific expression in transgenic rice seeds”, (1999) Plant Mol. Biol., 39(6): 1101-1112) which reported that GUS expression peaked at 41 DAP based on the staining density of GUS protein in rice endosperm. This difference could be due to the use of the complete Tapur signal peptide in our study, where this sequence was truncated in Digeon's work.

### EXAMPLE 7

### Improvement of Lysozyme Expression by Combining Tapur and Gt1 Expression Cassettes

Using the Tapur promoter and signal peptide for targeting, human lysozyme was delivered to both protein bodies I and II (FIG. 5) rather than rice starch granule. By targeting an organelle other than protein body II, using the Gt1 promoter and signal peptide (Yang, D., et al., “Expression and localization of human lysozyme in the endosperm of transgenic rice”, Planta, 2003. 216(4): 597-603), lysozyme expression improved in rice endosperm when combining both expression cassettes. As human lysozyme was stored in protein body I and II when driven by the Tapur cassette (FIG. 5), additive or synergistic effects on expression of human lysozyme could be obtained by targeting to different organelles using co-expression experiments. Two approaches were designed to test the hypothesis. One approach was to co-transform pAPI308 (Tapur-sig-lysozyme) and pAPI159 (Gt1-sig-lysozyme) onto non-transgenic TP309 calli. Resulting plants carrying integrated copies of both expression cassettes were designated as 159/308. The second approach, called gene stacking, was to bombard pAPI308 onto the calli derived from rice transgenic line 159-53, a stable and homozygous transgenic line with an expression level of 120 μg/grain (Huang et al., “Expression of functional recombinant human lysozyme in transgenic rice cell culture”, (2002) Transgenic Res, 11(3): 229-39; Yang et al., “Expression and localization of human lysozyme in the endosperm of transgenic rice”, (2003) Planta 216(4): 597-603). Plants resulting from this approach were designated as 308//159 (see Table 1). A total of 125 independent transgenic events from 159/308 and 148 independent transgenic events from 308//159 were generated. Of these 60 and 79 transgenic events were fertile from 159/308 and 308//159, respectively. The lysozyme content of seeds produced by these plants was assayed and compared to the results obtained when each cassette was transformed individually. The expression level of human lysozyme from 159/308 ranged from 22.2 μg/grain to 110.0 μg/grain averaging 56.1±28.1 μg/grain (Table 1). The overall expression levels were significantly higher than those produced by 159 alone, and the lines with highest expression level were remarkably higher than that of Gt1-Lys alone. The expression level of human lysozyme in 308//159 ranges from 58.4 μg/grain to 201.5 μg/grain, averaging 137.0±26.2 μg/grain. Bombardment of pAPI308 onto calli derived from line 159-53 resulted in transgenic plants with expression levels significantly higher than either construct produced independently (Table 1). Comparison of expression levels in the highest expressing lines and on average indicates an additive effect was obtained from both 308//159 and 308/159.

To confirm the additive effect, line 308//159-61, with an expression level of 169 μg/seed in R1 grain, was advanced to a second generation to monitor the expression level of R2 seed. The lysozyme level in R2 seed from 12 individual plants was assayed. Lysozyme content in 308//159-61 has a range of 106.3-202.4 μg/seed with an average of 140.4±27.8 μg/seed (Table 3). The data also suggests that genetic segregation occurred in the R1 generation. Five of the twelve lines had expression levels statistically equivalent to 159-53, indicating the transgene could be segregated out. Six lines produced significantly more lysozyme than 159-53, averaging 161.25 μg/seed (P<0.01). The best line, 308//159-61-13, expressed lysozyme at 202.4 μg/seed. These results demonstrate that simultaneously targeting human lysozyme to different cell compartments is a viable approach for increasing recombinant protein production in transgenic rice seeds.

TABLE 3
Statistical analysis of human lysozyme expression level in
308//159-61 R₂ seeds and 159-53 R₆ seeds

| Line # | Average activity ± S (n = 8), | t-Test (vs. 159-53) |
|---|---|---|
| 159-53 R6 | 120.00 ± 14.51 | Control |
| 308//159-61-1 | 106.31 ± 11.54 | 7.5 × 10⁻²** |
| 308//159-61-2 | 114.53 ± 16.56 | 0.50 |
| 308//159-61-3 | 123.19 ± 16.15 | 0.68 |
| 308//159-61-4 | 126.42 ± 11.86 | 0.34 |
| 308//159-61-5 | 142.82 ± 9.76 | 2.2 × 10⁻³** |
| 308//159-61-6 | 122.72 ± 11.11 | 0.68 |
| 308//159-61-7 | 151.07 ± 9.36 | 2.3 × 10⁻⁴** |
| 308//159-61-8 | 143.28 ± 12.30 | 04.3 × 10⁻³** |
| 308//159-61- | 124.15 ± 15.99 | 0.60 |
| 308//159-61- | 148.16 ± 10.47 | 5.49 × 10⁻⁴** |
| 308//159-61- | 179.82 ± 19.26 | 6.08 × 10⁻⁶** |
| 308//159-61- | 202.37 ± 12.45 | 7.68 × 10⁻⁹** |

All publications cited herein are incorporated herein by reference for the purpose of describing and disclosing terminology, compositions and methodologies that might be used in connection with the invention.

### Brief Description of the Codon Optimized Nucleic Acid Sequences

| | SEQ |
|---|---|
| | ID |
| Description | NO |
| Pfu DNA polymerase reverse primer | 1 |
| 5′-GGGAATATTGTACCAGCCGCCAACTTCTGA-3′ | |
| Pfu DNA polymerase forward primer | 2 |
| 5′-CCGCTGCAGCTCCAACATCTTATCGCAACATCC-3′ | |
| Codon optimized lysozyme coding sequence: | 3 |
| AAAGTCTTCGAGCGGTGCGAGCTGGCCCGCACGCTCAAGCGGCTCGGCAT | |
| GGACGGCTACCGGGGCATCAGCCTCGCCAACTGGATGTGCCTCGCCAAGT | |
| GGGAGTCGGGCTACAACACCCGCGCAACCAACTACAACGCCGGCGACCGC | |
| TCCACCGACTACGGCATCTTCCAGATCAACTCCCGCTACTGGTGCAACGAC | |
| GGCAAGACGCCCGGGGCCGTCAACGCCTGCCACCTCTCCTGCTCGGCCCT | |
| GCTGCAAGACAACATCGCCGACGCCGTCGCGTGCGCGAAGCGCGTCGTCC | |
| GCGACCCGCAGGGCATCCGGGCCTGGGTGGCCTGGCGCAACCGCTGCCA | |
| GAACCGGGACGTGCGCCAGTACGTCCAGGGCTGCGGCGTCTGA | |
| Amino acid sequence based on codon optimized lysozyme coding | |
| sequence: | |
| KVFERCELARTLKRLGMDGYRGISLANWMCLAKWESGYNTRATNYNAGDRST | |
| DYGIFQINSRYWCNDGKTPGAVNACHLSCSALLQDNIADAVACAKRVVRDPQGI | |
| RAWVAWRNRCQNRDVRQYVQGCGV | |
| Gt1 promoter sequence | 4 |
| CATGAGTAATGTGTGAGCATTATGGGACCACGAAATAAAAAGAACATTTTGAT | |
| GAGTCGTGTATCCTCGATGAGCCTCAAAAGTTCTCTCACCCCGGATAAGAAA | |
| CCCTTAAGCAATGTGCAAAGTTTGCATTCTCCACTGACATAATGCAAAATAAG | |
| ATATCATCGATGACATAGCAACTCATGCATCATATCATGCCTCTCTCAACCTA | |
| TTCATTCCTACTCATCTACATAAGTATCTTCAGCTAAATGTTAGAACATAAACC | |
| CATAAGTCACGTTTGATGAGTATTAGGCGTGACACATGACAAATCACAGACT | |
| CAAGCAAGATAAAGCAAAATGATGTGTACATAAAACTCCAGAGCTATATGTCA | |
| TATTGCAAAAAGAGGAGAGCTTATAAGACAAGGCATGACTCACAAAAATTCA | |
| CTTGCCTTTCGTGTCAAAAAGAGGAGGGCTTTACATTATCCATGTCATATTGC | |
| AAAAGAAAGAGAGAAAGAACAACACAATGCTGCGTCAATTATACATATCTGTA | |
| TGTCCATCATTATTCATCCACCTTTCGTGTACCACACTTCATATATCATAAGA | |
| GTCACTTCACGTCTGGACATTAACAAACTCTATCTTAACATTTAGATGCAAGA | |
| GCCTTTATCTCACTATAAATGCACGATGATTTCTCATTGTTTCTCACAAAAAG | |
| CGGCCGCTTCATTAGTCCTACAACAAC | |
| Gt1 signal sequence | 5 |
| ATGGCATCCATAAATCGCCCCATAGTTTTCTTCACAGTTTGCTTGTTCCTCTT | |
| GTGCGATGGCTCCCTAGCC | |
| Purindoline promoter sequence | 6 |
| AAGCTTGCATGCCTGCAGAATGCCAGAATAAGAGGGGGAGAAGCTAGTCCT | |
| ATCAAAGACTACGCTTCCAGTAACCTCCGTCTCGCAGTAGTAGAAGAGAATA | |
| GCAGATAAGTATCAACACATAGCATAACCCACCTGGCGATCCTCTCCTTGTC | |
| ACCCTGTGAGAGAGCGAACACCGGGTTGTATCTGGAAGTTATCTGGGTGTG | |
| CTTTATTAAGTCGGCTGGTACATCATCCTCCCATAGGAGGCCTTTGCATCTG | |
| GGCGTGTGTGGCCTATTTTCATTTCACCCCAGTTATTCCATCGAACTAAGTA | |
| GCAACATGTAAGGAGTCAGTTTTCGAGATACCACACAACACCAATTTTCCAA | |
| CGAAACTAATGAGAAATAAAAAGGTGCATCACTCATTTTCGACCAAATTAATT | |
| ATGTCTTGGTATTAGAGTTTTCTCTCTCTGTCCTGATAAACCCAAACGGAGGA | |
| GTAAAGATTATCTATCTCAACATCACATGATTCTAAATACAAAACAGAAAACC | |
| ACGGCTAGAAGAGGACGACATCTAGAGGCATTGCTTTTCATGTACTAATACC | |
| TTGTTAAACACATTCTCTAACAAATTGGTTTGGATCCTTCTTCAACAATTTCCA | |
| CACACTACAAGGCCAGTTCACAAAAGCTTAAAGCGTGAGCATTGGTACAAAA | |
| CTAGTTGTGGTCTATCTTGAGAAAAGGGAACACTTAGTACACGAAACGTCAC | |
| CTGTCTCAACAACTTGCACCATTTCTGTTGGCTCGCAAAGTAACTTTATTTAG | |
| TATACCAACTTAATTTGTGAGCATTAGCCAAAGCAACACACAATGGTAGGCA | |
| AAAACCATGTCACTAAGCAATAAATAAAGGGGAGCCTCAACCCATCTATTCAT | |
| CTCCACCACCACCAAAACAACATTGAAAAC | |
| Purindoline signal sequence | 7 |
| ATGAAGACCTTATTCCTCCTAGCTCTCCTTGCTCTTGTAGCGAGCACAACCTT | |
| CGCGCAATACTCAGAAGCTGGCGGCTGGTACAAT | |

What is claimed is:

1. A method of producing seeds of a monocot plant that accumulate a heterologous protein, which method comprises the following steps:
(a) stably transforming a plant cell of the monocot plant with a chimeric gene to obtain a transformed monocot plant cell, the chimeric gene comprising
(i) a promoter from a monocot non seed-storage protein gene,
(ii) a first DNA sequence, operably linked to said promoter, encoding a monocot seed-specific signal peptide capable of targeting a linked polypeptide to an intracellular region within a seed cell, and
(iii) a second DNA sequence, operably linked to said promoter and linked in translation frame with the first DNA sequence, encoding the heterologous protein, wherein the first DNA sequence and the second DNA sequence together encode a fusion protein comprising the signal peptide and heterologous protein;
(b) growing a plant from the transformed plant cell to produce seeds that express the heterologous protein; and
(c) harvesting the seeds from the plant grown in step (b) to obtain the seeds that accumulate the heterologous protein.

2. The method of claim 1, wherein the monocot seed-specific signal peptide is a monocot seed-specific N-terminal signal peptide.

3. The method of claim 1, wherein the monocot plant is a rice plant.

4. The method of claim 1, wherein the intracellular region is a protein body I, protein body II, starch granule, chloroplast, mitochondria or endoplasmic reticulum.

5. The method of claim 1, wherein the heterologous protein is an animal protein.

6. The method of claim 5, wherein the animal protein is a mammalian protein.

7. The method of claim 6, wherein the mammalian protein is a human protein.

8. The method of claim 7, wherein the human protein is selected from the group consisting of a blood protein, milk protein, human gastrointestinal peptide, lipase, amylase, colony stimulating factor, cytokine, interleukin, integrin, T cell receptor, immunoglobulin, growth factor and growth hormone of human origin.

9. The method of claim 8, wherein the human protein is selected from the group consisting of lysozyme, lactoferrin, lactoperoxidase, kappa-casein, hemoglobin, alpha-1-antitrypsin, fibrinogen, antithrombin III, serum albumin, trypsinogen, aprotinin, transferrin, growth hormone, antibody, insulin, insulin-like growth factor, epithelial growth factor, intestinal trefoil factor, granulocyte colony-stimulating factor and macrophage colony-stimulating factor of human origin.

10. The method of claim 1, wherein the promoter is a promoter of a gene selected from the group consisting of wheat purindoline b protein gene, protein disulfide isomerase gene and heat shock 70 protein gene.

11. A method of producing a substantially purified protein heterologous to a monocot plant, comprising the method of claim 1, and further comprising processing the seeds to obtain a fraction enriched for the heterologous protein, and purifying the heterologous protein from the enriched fraction to obtain the protein heterologous to the monocot plant.

12. A method of producing seeds of a monocot that accumulate a heterologous protein in at least two intracellular regions within a cell of the seeds of the monocot, which method comprises the steps of:
(a) stably co-transforming a cell of the monocot with at least first and second chimeric genes to obtain a transformed monocot cell, the first chimeric gene comprising
(i) a first promoter from a monocot protein gene,
(ii) a first DNA sequence, operably linked to the promoter, encoding a first monocot seed-specific signal peptide capable of targeting a polypeptide linked thereto to a first intracellular region within a monocot seed cell, and
(iii) a second DNA sequence, operably linked to the first promoter and linked in translation frame with the first DNA sequence, encoding the heterologous protein, wherein the first and second DNA sequences together encode a fusion protein comprising the first monocot seed-specific signal peptide and the heterologous protein,
the second chimeric gene comprising
(i) a second promoter from a monocot protein gene,
(ii) a third DNA sequence, operably linked to the promoter, encoding a second monocot seed-specific signal peptide capable of targeting a polypeptide linked thereto to a second intracellular region within a monocot seed cell, and
(iii) a fourth DNA sequence, operably linked to the second promoter and linked in translation frame with the third DNA sequence, encoding the heterologous protein, wherein the third and fourth DNA sequences together encode a fusion protein comprising the second monocot seed-specific signal peptide and the heterologous protein,
wherein the first and second promoter are different, the first and second monocot seed-specific signal peptides are different, and the first and second intracellular regions are different;
(b) growing a monocot plant from the transformed monocot cell to produce seeds that express the heterologous protein in at least two different intracellular regions; and
(c) harvesting the seeds from the monocot plant grown in step (b) to obtain the seeds of the monocot that accumulate the heterologous protein.

13. A method of producing seeds of a monocot that accumulate a heterologous protein in at least two different intracellular regions within a cell of the seeds of the monocot, which method comprises the steps of:
(d) stably transforming a first cell of the monocot with a first chimeric gene to produce a first transformed cell of the monocot, the first chimeric gene comprising
(i) a first promoter from a monocot protein gene,
(ii) a first DNA sequence, operably linked to the first promoter, encoding a first monocot seed-specific signal peptide capable of targeting a polypeptide linked thereto to a first intracellular region within a monocot seed cell, and
(iii) a second DNA sequence, operably linked to the first promoter and linked in translation frame with the first DNA sequence of (a)(ii), encoding the heterologous protein, wherein the first and second DNA sequences together encode a fusion protein comprising the first monocot seed-specific signal peptide and the heterologous protein;
(e) stably transforming a second cell of the monocot with a second chimeric gene to produce a transformed second cell of the monocot, the second chimeric gene comprising
(i) a second promoter from a monocot protein gene,
(ii) a third DNA sequence, operably linked to the second promoter, encoding a second monocot seed-specific signal peptide capable of targeting a polypeptide linked thereto to a second intracellular region within a monocot seed cell, and
(iii) a fourth DNA sequence, operably linked to the second promoter and linked in translation frame with the third DNA sequence of (b)(ii), encoding the heterologous protein, wherein the third and fourth DNA sequences together encode a fusion protein comprising the second monocot seed-specific signal peptide and the heterologous protein,
wherein the first and second promoter may be the same or different, the first and second monocot seed-specific signal peptides are different, and the first and second intracellular regions are different;
(f) growing a monocot plant from the first transformed cell of (a) to produce a first monocot plant that can express the heterologous protein in the first intracellular region;
(d) growing a monocot plant from the second transformed cell of (b) to produce a second monocot plant that can express the heterologous protein in the second intracellular region;
(e) crossing the first and second monocot plants to produce a hybrid plant;
(f) growing the hybrid plant to produce seeds that can express the heterologous protein in the first and second intracellular regions in the same seed cell; and
(g) harvesting the seeds from the hybrid plant to obtain the seeds of the monocot that accumulate the heterologous protein.
